(19) United States
(12) Patent Application Publication
(10) Pub. No.: US 2021/0338047 A1
(43) Pub. Date: Nov. 4, 2021

(54) A POWER-ADAPTABLE DEVICE FOR SCANNING A HUMAN INTRA-CAVITY

(71) Applicant: 3SHAPE A/S, Copenhagen K (DK)

(72) Inventors: Kasper Krogh HANSEN, Copenhagen Ø (DK); Troels Böwadt ARNVED, Virum (DK); Frederik JUUL, Copenhagen K (DK)

(73) Assignee: 3SHAPE A/S, Copenhagen K (DK)

(21) Appl. No.: 17/280,375

(22) PCT Filed: Sep. 24, 2019
(86) PCT No.: PCT/EP2019/075680

§ 371 Date: Mar. 26, 2021

(30) Foreign Application Priority Data
Sep. 27, 2018 (EP) 18197090.6

(51) Int. Cl. A61B 1/00; A61B 1/227; A61B 1/24

(52) U.S. Cl. CPC A61B 1/00029; A61B 1/00016; A61B 1/00018; A61B 1/00009; A61B 1/227; A61B 1/24; A61B 1/00032

(57) ABSTRACT

The present disclosure provides in a first aspect of the invention a device for scanning a human intra-cavity, comprising a housing comprising a part connected to the housing, the part dimensioned for being inserted into said human intra-cavity, a single mounting-interface on the housing, the single mounting-interface configured to removably mount an internal power supply unit for powering the device from an internal power supply within the internal power supply unit, or an external power supply unit for powering the device from an external power supply outside the external power supply unit, the device configured for being changed between two power-operation modes.

### FIELD OF THE INVENTION

This invention generally relates to adaptable powering of scanning devices, more specifically to adaptable powering of devices for scanning a human intra-cavity, for example an intra-oral cavity, or an intra-ear cavity.

### BACKGROUND

In the field of scanning human intra-cavities, such as in the field of scanning teeth, it has been common practice for a long period of time to use a cable-powered scanner. Only recently, human intra-cavity scanners have been made wireless.

Thus, scanning devices are known to be powered either via a cable or via a battery. Today, such scanning devices are manufactured and sold.

Today, it is up to the end-user to decide whether he or she wants to buy and operate either a cable-powered scanning device or a battery-powered scanning device.

In most situations, the end-user prefers to buy and operate a battery-powered scanning device because it provides a flexible handling without a cable in the way and the opportunity to share a scanning device between multiple rooms or chairs.

However, in some situations, the battery-powered scanning device may run out of power because scanning is a power-demanding task. In non-preferred situations, the battery-powered scanning device may even run out of power during scanning.

One solution to the non-preferred situation may be to charge the battery-powered scanning device, but this takes time, and scanning cannot proceed immediately. Accordingly, this solution may result in the most unwanted scenario with no available power source to operate the scanning device. Instead of charging the battery-powered scanning device, it may be possible to charge one or more batteries of the battery-powered scanning device, and then replace the out-run-battery with a fully charged battery. This takes time, and an alternative solution is to replace the entire battery-powered scanning device with a fully charged battery-powered scanning device. This solution is of course non-optimal in terms of cost.

Another solution to the non-preferred situation may be to power the scanning device by plugging a power cable into the scanning device, for example by powering the scanning device by a power cable plugged into a power-inlet in the scanning device.

Another way of powering a scanning device may for example be by providing power over a USB-connection. In such cases, the user may plug a USB-cable with a USB-plug into a USB-slot on the scanning device. A USB-slot is commonly used on devices with an integrated battery, i.e. a battery that is build-in the device. Thus, it is known that devices with a USB-slot can be powered via a USB-plug connected to a USB-cable as connected to for example a computer. Alternatively, such devices with a USB-slot can be powered via a USB-plug connected to a USB-cable as connected to for example a power bank.

Smart phones with an integrated battery and a USB-slot are examples of devices with integrated batteries.

In devices with an integrated battery, power from a given power supply is transferred via the integrated battery, whereby the device gets charged.

Several problems are related to powering devices with integrated batteries.

For example, an integrated battery takes up space in a scanning device, making the scanning device bulky. Further, an integrated battery adds unnecessary weight to the scanning device when being operated in a cable-powered manner. An integrated battery suffers from tedious recharging time, as an easy and simple replacement of the depleted battery is not possible. Even further, the widely used Li-ion battery technology used as integrated batteries are associated with safety hazards, such as risk of temperature run-away which can cause explosion or fire, for example during transportation. Last, but not the least, an integrated battery adds an additional cost to the scanning device.

Further, devices, such as scanning devices, with an integrated battery and a standard USB-slot, also have another problem. Using a USB-connection via a standard USB slot to power the scanning device is associated with a harmful risk, since a USB-cable can be connected to consumer grade equipment and thus fail to provide the high voltage isolation required by the medical safety standard. A USB-wired device to acquire intraoral 2D-images is described in US 2013/0203010.

Also, a USB-slot on the scanning device may be an additional slot to for example a power-inlet, which may then add material, weight and cost to the scanning device. Additionally, a USB slot may cause unnecessary cavities in the scanner housing may be difficult to clean and may result in hygienic problems.

All in all, several disadvantages are associated with known scanning devices that can scan in a battery-powered manner or in wired manner.

Accordingly, an improved scanning device is desired.

### SUMMARY

An objective of the present invention is to overcome the before-mentioned disadvantages.

### First Aspect of the Invention

The present disclosure provides in a first aspect of the invention a device for scanning a human intra-cavity, comprising: a housing comprising a part connected to the housing, the part dimensioned for being inserted into said human intra-cavity, a single mounting-interface on the housing, the single mounting-interface configured to removably mount: (i) an internal power supply unit for powering the device from an internal power supply within the internal power supply unit, or (ii) an external power supply unit for powering the device from an external power supply outside the external power supply unit, the device configured for being changed between two power-operation modes: (i) an internal power-operation mode, where the device is powered by the internal power supply via the single mounting-interface, and (ii) an external power-operation mode, where the device is powered by the external power supply via the single mounting-interface.

The present invention as here described provides a scanning device that overcomes the before-mentioned disadvantages.

The present invention overcomes the before-mentioned disadvantages by providing a device that at least:
(i) has a single interface for an external power supply unit or the internal power supply unit;
(ii) removably mounts the external power supply unit or the internal power supply; and
(iii) directly powers the device using the external power supply unit or the internal power supply unit.

Furthermore, the present invention provides in relation to the first aspect that the device is further configured for being changed between two data-operation modes: a wireless data-operation mode, where the device transfers data in a form of wireless data signals to a wireless module, and a wired data-operation mode, where the device transfers data in a form of wired data signals to the single mounting-interface.

This present invention allows the end-user to select how to transfer data. For example, in some situations, where a wireless network is not accessible, and/or where the device has limited connectivity and/or where the wireless network is of limited speed, the end-user might not be able to scan using a wireless network. Further, in other situations, a wireless network may be prohibited from being used. The present invention provides the end-user to change from wireless data-operation mode to wired data-operation mode, and thus allows the end-user to scan a human intra-cavity using the herein disclosed device.

By transferring wired data signals to the single mounting-interface, there must be some means of further transmitting the wired data signals to a for example a base station. Accordingly, to provide the base station with data in a wired manner, either the internal power supply unit or the external power supply unit needs to be configured to transfer wired data signal via a wire to the base station. This will be discussed under details of the internal and external power supply units. However, because the wired data signals are transferred to the single mounting-interface, and for example not to an additional interface, this embodiment has the advantage of providing a compact and low-cost scanning device for transferring both power and data.

Even further, the present invention provides in relation to the first aspect that the device comprises a host controller. The host controller is configured to transfer the data in both the wireless data-operation mode and the wired data-operation mode. In other words, the host controller is responsible for transferring the data in both the wireless data-operation mode and the wired data-operation mode. Typically, a host controller is only used in portable devices to transfer data in a wireless mode, not in a wired mode. When a portable device transfers data in a wired mode, the portable device typically bypasses the host controller, and instead uses a device controller that is not set-up as a host. Instead, a computer that connects to the portable device has the host controller, whereby the computer always will act as host for the portable device.

An example of a typical set-up between a computer (PC) and an embedded device is shown in FIG. 7. The set-up in FIG. 7 uses a USB-cable between the PC and the embedded device. In such a typical set-up for a wired connection to a PC, the embedded device does not comprise the host controller—it is located only in the PC.

If the embedded device communicates over WiFi (USB to WiFi), the embedded device may comprise a host controller, and such a set-up is shown exemplified in FIG. 8.

FIGS. 7-8 show examples of the typical prior art set-up between an embedded device and a PC using a USB connection.

As above described, the present invention differs from the prior art in that the host controller is configured to transfer the data in both the wireless data-operation mode and the wired data-operation mode.

An example of a set-up that shows how the present invention works in the wired data-operation mode using the host-controller is shown in FIG. 9.

By configuring the scanning device with the controller to transfer the data in both the wireless data-operation mode and the wired data-operation mode, it becomes possible to obtain a single interface that is both compact and provides for wired connection with high voltage isolation required by the medical safety standard. Accordingly, the present invention overcomes the problems as present in prior art medical devices using USB connections.

Other advantages and embodiments will be discussed in the following.

### Single Interface

According to the first aspect of the invention, the single mounting-interface is configured to mount the internal power supply unit or the external power supply unit.

Thus, the end-user can buy the device without immediately deciding on whether he or she wants a scanning device with an internal power supply unit or with an external power supply unit. The end-user can in principle acquire the device without any of the two power supply units, and then later figure out which power supply is desired. The end-user may however want to operate the scanning device by acquiring one of the two power supply units. For example, the end-user may as a starting point want to acquire the scanning device with an external power supply unit. At a later point, the end-user may want to use the scanning device as powered by an internal power supply unit, and then at this later point, acquire the internal power supply unit.

An advantage of the present invention is that the end-user is free to choose the type of power supply that he or she wants to use.

Accordingly, by having a single mounting-interface, the first aspect of the present invention provides a very flexible scanning device.

Since there is only a single mounting-interface, and according to the invention thereby only means for removably mounting the internal or the external power supply unit, the invention does not relate to a device comprising two mounting-interfaces for two power supply units, such that both the internal power supply unit and the external power supply unit can be mounted at such two mounting-interfaces on the device at the same time.

Further, for the same reason as above, the invention does not relate to a scanning device comprising for example a battery compartment for mounting a battery and a power-inlet for connecting to an external power supply unit.

Finally, and for the same reason above, the invention does not relate to a scanning device with an integrated battery that is coupled to a mounting-interface on the device for connecting an external power supply.

Thus, by only having a single mounting-interface, the present invention provides a cost-efficient scanning device with a small form factor, low weight, and with no harmful risks.

### Removably Mounting of the Internal or External Power Supply Unit

According to the invention, the single mounting-interface is configured to removably mount the internal power supply unit or the external power supply unit.

Since the internal power supply unit is removably mounted to the device, the internal power supply unit is not an integrated power supply unit, such as for example an integrated battery.

Since the internal power supply is not an integrated power supply unit, the scanning device is alone not associated with the harmful risks of exploding.

Further, since the internal power supply is not an integrated power supply unit, the internal power supply does not necessarily add weight to the device when being used in the external operation-power mode.

Thus, by having the single mount configured to removably mount the internal power supply unit or the external power supply unit, the present invention provides a cost-efficient scanning device with a small form factor, low weight, and with no harmful risks.

### Direct Powering of the Device

According to the invention, the present invention directly powers the device using either an external power supply unit or an internal power supply unit.

The present invention does therefore not indirectly power the device by an external power supply or an internal power supply, for example by providing power via an integrated battery.

Since the internal power supply is not an integrated power supply unit, the scanning device is alone not associated with the harmful risks of exploding.

Further, since the internal power supply is not an integrated power supply unit, the internal power supply does not necessarily add weight to the device when being used in the external operation-power mode.

Thus, by directly powering the device, the present invention provides a cost-efficient scanning device with a small form factor, low weight, and with no harmful risks.

### Second Aspect of the Invention

The present disclosure provides in a second aspect the invention an internal power supply for a device according to the first aspect.

In one embodiment, the internal power supply unit is a battery pack.

In a related embodiment, the internal power supply unit is configured for being charged via inductive or capacitive coupling.

Regardless of the embodiment of the internal power supply unit, the internal power supply unit provides internal power to the scanning device via the single mounting-interface on the scanning device.

Further, regardless of the embodiment of the internal power supply unit, the device is also configured for being changed to receive power from the external power supply unit via the single mounting-interface.

For example, if the scanning device is powered by an internal power supply unit in the form of a battery that is mounted in the single mounting-interface, the device can be changed to receive power from the external power supply unit via the single mounting-interface.

One way of changing from being powered from the internal power supply unit to being powered from the external power supply may be to replace the internal power supply unit with an external power supply unit.

Another way of doing it, may be to provide a mounting-interface feed-through on the internal power supply unit, and then place an external power supply unit in the mounting-interface feed-through on the internal power supply unit. In this way, the single mounting-interface is still configured to mount an external power supply unit for powering the device from an external power supply outside the external power supply unit, according to the first aspect of the invention, and the device is still in the external power-operation mode, where the device is directly powered by the external power supply unit via the single mounting-interface, because the external power supply does not power an integrated battery. Furthermore, in some embodiments, the internal power supply unit may comprise a by-pass circuit such that in embodiments when connected with an external power supply unit, the internal power supply unit is not charged.

### Third Aspect of the Invention

The present disclosure provides in a third aspect of the invention an external power supply for a device according to the first aspect.

In one embodiment, the external power supply unit is configured to be wired to an external power supply.

In another embodiment, the external power supply unit is configured to receive power from an external power supply in a wireless manner, for example using wireless power transfer, such as using inductive coupling or capacitive coupling.

Regardless of the embodiment of the external power supply unit, the external power supply unit provides external power to the scanning device via the single mounting-interface on the scanning device.

Further, regardless of the embodiment of the external power supply unit, the device is also configured for being changed to receive power from the external power supply unit via the single mounting-interface.

For example, if the scanning device is powered by an external power supply unit in the form of a plug that is mounted in the single mounting-interface, where the plug having a wired connection to an external power supply, the device can be changed to receive power from the internal power supply unit via the single mounting-interface.

As another example, if the scanning device is powered by an external power supply unit in the form of a wireless power receiver that is mounted in the single mounting-interface, where the wireless power receiver having a wireless connection to an external power supply, the device can be changed to receive power from the internal power supply unit via the single mounting-interface.

One way of changing from being powered from the external power supply unit to being powered from the internal power supply may be to replace the external power supply unit with an internal power supply unit.

Another way of doing it, may be to provide a mounting-interface on the external power supply unit, and then place an internal power supply unit in the mounting-interface on the external power supply unit.

In this way, the single mounting-interface is still configured to mount an internal power supply unit for powering the device from an internal power supply inside the external power supply unit, according to the first aspect of the invention, and the device is still configured to be in the external power-operation mode, where the device is directly powered by the internal power supply unit via the single mounting-interface, because the internal power supply does not power an integrated battery.

### Fourth Aspect of the Invention

The present disclosure provides in a fourth aspect the invention an intra-cavity scanning system, comprising: the device according to the first aspect of the invention; and the internal power supply unit according to the second aspect of the invention; and/or the external power supply unit according to the third aspect of the invention.

As previously described, the end-user is free to choose which of the power supply-units he or she desires.

The present invention allows an end-user to for example acquire the scanning system according the fourth aspect of the invention, particularly a single scanning device that is fit for purpose, for example either as a wired scanner or a wireless scanner. The user may for example choose to acquire the single scanner device as set up in the external power-mode, i.e. with the external power supply unit, but without buying the internal power supply unit.

Alternatively, the user may for example choose to acquire the single scanner device as set up in the internal power-mode, i.e. with the internal power supply unit, but without acquiring the external power supply unit.

By the present invention, the user may as described above choose to acquire the single scanner in the one mode, or in the other mode.

Regardless of how the end-user acquires the single scanner, the end-user may at his or her desire choose to upgrade the single scanner, such that the scanner may change from the one mode to the other mode or the other way around.

Of course, the user may also choose to acquire the scanning device with both the internal power supply unit and the external power supply unit, and then change between the two modes as desired, for example depending on the scanning task and/or the scanning environment.

### BRIEF DESCRIPTION OF THE DRAWINGS

FIG. 1 shows an example of a scanning device with two different power supply configurations.

FIG. 2 shows an example of the single mounting-interface in the scanning device.

FIG. 3 shows an example of an internal power supply unit for the scanning device.

FIGS. 4a-4b shows an example of an external power supply unit for the scanning device.

FIG. 5 shows an example of a hybrid internal- or external power supply unit.

FIGS. 6a-6c shows an example of an alternative configuration of a scanning device and power supply units.

FIG. 7 shows an example of prior art of wired communication with a USB-cable between an embedded device and a PC.

FIG. 8 shows an example of wireless communication with USB to WiFi between an embedded device and a PC.

FIG. 9 shows an example of how the scanning device according to the present invention communicates over a wire between the scanning device (the embedded device and a PC) using a host controller located on the scanning device.

### DETAILED DESCRIPTION

As previously described, the device is configured for being changed between two power-operation modes. These power-operation modes are preferably modes where the device is in operation, i.e. where the device is scanning. The invention provides further operation modes as will be discussed in the following.

### Operation Modes

In one embodiment, the device is further configured for providing any combination of the two power-operation modes and the two data-operation modes.

Specifically, when in the internal power-operation mode, the end-user may select between the wireless data-operation mode and the wired data-operation mode. Also, when in the external power-operation mode, the end-user may select between the wireless data-operation mode and the wired data-operation mode. To select between the two modes, the end-user may select one of the two data-operation modes by software or by a switch on the device or on the power supply unit. In other words, the device may in some embodiment comprise a switch configured to select between the two data-operation modes.

In a preferred embodiment, the device is further configured for being changed between two fully-operation modes: a fully wireless-operation mode, where the device is in the internal power-operation mode and the wireless data-operation mode, and a fully wired-operation mode, where the device is in the external power-operation mode and the wired data-operation mode.

In this embodiment, the device may further be configured for being automatically changed between the two fully-operation modes based on what is mounted in the single mounting-interface. For example, when the internal power supply unit is mounted in the single mounting-interface, the device is configured to detect that the internal power supply unit is mounted, and to automatically change to the wireless data-operation mode. In addition, when the external power supply unit is mounted in the single mounting-interface, the device is configured to detect that the external power supply unit is mounted, and to automatically change to the wired data-operation mode. When the device is operated in the fully wireless operation mode and the internal power supply unit is mounted in the single mounting-interface, the scanner may await the operator to manually turn on the scanner in order to conserve battery until the operator is ready to scan. When the scanner is operated in the fully wired operation mode, the scanner may automatically start up, when detecting the external power supply unit, and for example stand by in an idle state until the operator is ready to scan.

### The Single Mounting-Interface

In one embodiment, the single mounting-interface is a slot or a socket. In another embodiment, the single mounting-interface is a plug.

In a preferred embodiment, the single mounting-interface comprises: at least one data coupler, at least one voltage coupler, and at least one detector coupler. In a most preferred embodiment, the single mounting-interface comprises one data coupler pair, one voltage coupler pair, and one detector coupler. In some embodiments, one or more of the coupler(s) is/are in general in the form of a contacting member configured to match with a contacting member on internal power supply unit or the external power supply unit. In some embodiments, the one or more coupler(s) is/are in the form of a pin and/or a wire. In some embodiments, the one or more coupler(s) is/are one or more male and/or female contacting member(s). For example, the single mounting interface may be a socket with a plurality of pins, such as between 4 and 10 pins, preferably between 6 and 8 pins, most preferably around 7 or 8 pins. In some embodiments, the detector coupler is configured for detecting the presence of either the internal power supply unit or the external power supply unit. In some embodiments, the detector coupler is a thermistor coupler, such as a thermistor pin, whereby the temperature of the internal power supply unit can be detected in the device. In some embodiments, by detecting the temperature, the device is thereby configured for detecting the presence of an internal- or external power supply unit. For example, the temperature may be detected via measuring the voltage and/or the electrical resistance of the thermistor coupler by a thermistor voltage divider coupled to the thermistor coupler.

In a more preferred embodiment, the single mounting-interface further comprises at least one universal asynchronous receiver-transmitter (UART) coupler pair or one synchronous serial interface (SSI) coupler pair. Such a pair may typically be used in production, for example for testing or in the field, for example for communicating with a battery fuel gauge integrated circuit. The coupler pairs may be configured to operate as either UART or I2C dependent of the operation mode of the device. When operated in the UART mode, the coupler pair may be configured as RX and TX. When operated in I2C mode, the coupler pair may be configured as SDA and SCL.

In one embodiment, the single mounting-interface is configured for replaceably mounting the internal power supply unit or the external power supply unit. In a second embodiment, the single mounting-interface comprises a part that geometrically matches a part of the internal power supply unit and geometrically matches a part of the external power supply unit. For example, the single mounting-interface may be in the form of a socket, wherein the socket comprises a rim or compartment that geometrically matches an outer surface of a part of the internal power supply unit and also geometrically matches an outer surface of a part of the external power supply unit. The two above described embodiment allow both the internal power supply unit and the external power supply unit to fit and being fixed into the single mounting-interface. Thus, in some embodiments, the internal power supply unit can be replaced by the external power supply unit and the other way around.

In one embodiment, the single mounting-interface comprises a surface to magnetically couple to the internal power supply unit or the external power supply unit. This embodiment allows that the internal power supply unit or the external power supply unit can be held tightly in place and provides the end-user with an auditive feedback, such as a click sound, when either the internal- and/or external power supply unit(s) is correctly and firmly secured in the single mounting-interface.

In another embodiment, the single mounting-interface comprises a mechanical locking mechanism to couple to the internal power supply unit and/or the external power supply unit. This embodiment allows that the internal power supply unit or the external power supply unit be held securely in place. For example, the mechanical locking mechanism may be a hole configured to match a rod, or a rod configured to match a hole. If combined with the embodiment with a surface to magnetically couple to the internal power supply unit or the external power supply unit, the combination provides for a very secure attachment of the power supply unit(s). For example, it enables the end-user to be able to grab the device by the cord (in the external power operation mode) if the device by accident is dropped, without damaging or braking circuits, wires or solders.

### Acquisition Unit and Processing Unit

In one embodiment, the device further comprises an acquisition unit configured for acquiring raw data of the human intra-cavity. For example, the acquisition unit may be an image sensor. The image sensor may be optically coupled to an imaging system in the device, for example an imaging system comprised of a plurality of lenses. In some embodiments, the imaging system may comprise a moving lens to provide scanning of the human-intra cavity. In other embodiments, the imaging system may comprise a focus changing element, such as a wavelength-dependent unit configured to change the wavelength of light being emitted from a light source inside the device and/or from a light source in the part dimensioned for being inserted into the human intra-cavity.

In a first related embodiment, the device further comprises a first processing unit linked to the acquisition unit and configured to process the raw data into processed data. In some embodiments, the first processing unit is an integrated circuit. For example, such a first processing unit may be in the form of a field-programmable gate array (FPGA).

In a second related embodiment, the device further comprises a second processing unit coupled to the acquisition unit and/or coupled to the first processing unit and linked to the wireless module and the single mounting-interface such that the processed data is sent to the wireless module in the wireless data-operation mode and the processed data is sent to the single mounting-interface in the wired data-operation mode. In some embodiments, the second processing unit is a central processing unit (CPU) comprising a reduced instruction set computer (RISC) architecture. For example, the second processing unit may be in the form of an Advanced RISC Machines (ARM)-processor such as based on 32 bits or 64 bits instructions.

In a preferred embodiment, the first processing unit and/or the second processing unit is configured to run software allowing the device to act as a host for the wireless module and to act as a host for an additional device in the external power supply unit, for example, the additional device may be a USB-to-Ethernet converter. In some embodiments, the software is defined to be a host controller driver.

In most embodiments, the host controller, together with a host controller driver (to be run by the first and/or second processing unit), acts as a host for the wireless module and acts as a host for an additional device in the external power supply unit. In most preferred embodiments, the host controller is a USB host controller.

### Wireless Module

In one embodiment, the wireless module is located in the internal power supply unit.

In a preferred embodiment, the wireless module is located in the scanning device connected to first and/or second processing unit.

In a second embodiment, the device is further configured to disable the wireless module upon the external power supply unit being mounted in the single mounting-interface and/or upon the internal power supply unit being dismounted the from the single mounting-interface. This allows for automatic setup, thereby providing a user-friendly device.

In a third embodiment, the device is further configured to enable the wireless module upon the internal power supply unit being mounted in the single mounting-interface and/or upon the external power supply unit being mounted in the single mounting-interface. This allows for automatic setup, thereby providing a user-friendly device.

In yet another embodiment, the wireless module is based on 802.11a/b/g/n/ac/ad/af (WI-FI), Bluetooth/Bluetooth Low Energy (BLE), or Worldwide Interoperability for Microwave Access (WiMax) technology.

### The Internal Power Supply Unit

In one embodiment, the internal power supply unit is a battery pack comprising at least one battery cell.

In another embodiment, the internal power supply is a small compact media for storing electrical, mechanical or chemical energy, such as a solid oxide fuel cell (SOEC).

In a third embodiment, the internal power supply unit comprises: at least one voltage coupler, preferably a voltage coupler pair, and at least one detector coupler. The detector coupler is in one embodiment as thermistor coupler, such as a thermistor pin, whereby the temperature of the internal power supply unit can be detected in the device.

In yet another embodiment, the internal power supply unit further comprises at least one universal asynchronous receiver-transmitter coupler pair.

In one embodiment, the internal power supply comprises a surface to magnetically couple to the single mounting-interface.

In another embodiment, the internal power supply unit comprises an interface to mount the external power supply unit.

In some embodiments, the internal power supply unit comprises a wireless module.

### The External Power Supply Unit

In one embodiment, the external power supply unit comprises: at least one data coupler, at least one voltage coupler, and at least one detector coupler.

In a preferred embodiment, the external power supply unit comprises one data coupler pair, one voltage coupler pair, and one detector coupler.

In a related embodiment, the external power supply unit further comprises at least one universal asynchronous receiver-transmitter coupler pair.

The external power supply unit may typically be a in form of plug of a wire. The wire may connect to the external power supply.

In some embodiments, the wire may be configured for transferring signals over Gigabit Ethernet, Fiber Channel, FireWire, PCI express or any Universal Serial Bus (USB) type.

In a preferred embodiment the external power supply unit comprises an additional device, wherein the additional device is a USB-to-Ethernet converter, such that an Ethernet cable is mountable in the USB-to-Ethernet converter. As few couplers in the mounting interface as possible is advantageous because the mounting interface can then be made as compact as possible. By having USB instead of ethernet saves 6 couplers. Thus, the USB-ethernet converter in the external power supply provides the solution for a compact design of the mounting interface in the device for scanning a human intra-cavity.

In one embodiment, the external power supply unit comprises a surface to magnetically couple to the single mounting-interface.

In a second embodiment, the external power supply unit comprises a locking mechanism to couple to the single mounting-interface. Examples of such mechanical locking mechanism could be a bayonet connector, spring interlock connector or friction connector.

In some embodiments, the external power supply comprises an interface to mount the internal power supply unit.

### Example 1—A First Embodiment of the Device

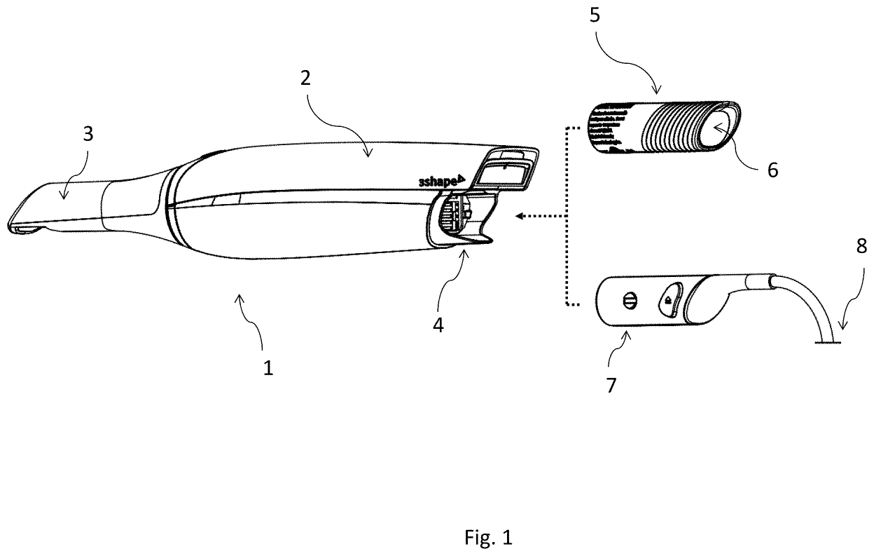

FIG. 1 show an example of the device according to the first aspect of the invention. FIG. 1 shows a device 1 for scanning a human intra-cavity, comprising: a housing 2 comprising a part 3 connected to the housing 2, the part 3 dimensioned for being inserted into said human intra-cavity, a single mounting-interface 4 on the housing 2, the single mounting-interface 4 configured to mount: an internal power supply unit 5 for powering the device 1 from an internal power supply 6 within the internal power supply unit 5, or an external power supply unit 7 for powering the device from an external power supply 8 outside the external power supply unit 7, the device configured for being changed between two power-operation modes: an internal power-operation mode, where the device 2 is directly powered by the internal power supply unit 6 via the single mounting-interface 4, and an external power-operation mode, where the device 1 is directly powered by the external power supply unit 7 via the single mounting-interface 4. When the internal power supply unit 5 is inserted in the single mounting-interface 4, the device 1 detects the voltage level of the internal power supply 5, thereby identifying the presence of the internal power supply 5.

### Example 2—A Single Mounting Interface

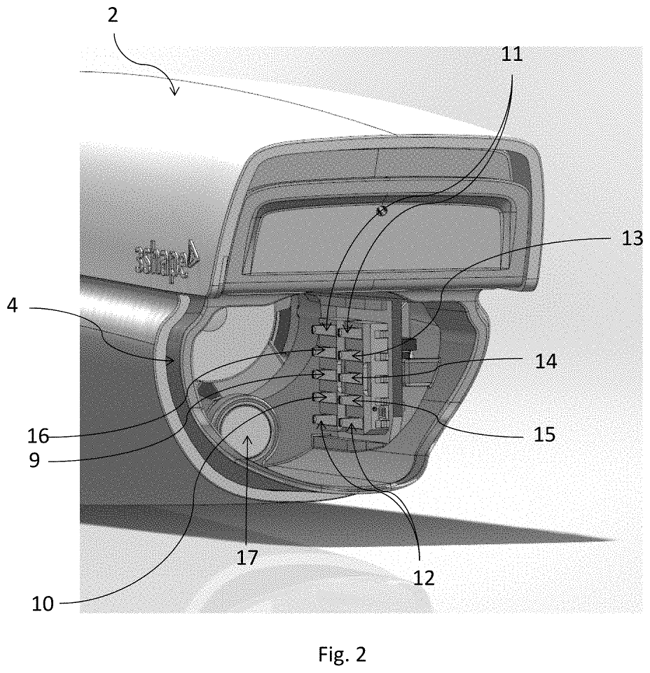

FIG. 2 illustrates an example of the single mounting interface 4. In this example, the single mounting-interface 4 is located in the housing 2 and comprises two data couplers 9 and 10. The two data couplers 9 and 10 are configured for communication through differential signaling utilizing USB signals. In the top of the single mounting-interface 4, there are two voltage couplers 11, and in the bottom of the single mounting interface, there are two additional voltage couplers 12. The voltage couplers 11 are electrically connected to operate as a single voltage coupler. Further, the additional voltage couplers 12 are electrically connected to operate as a single additional voltage coupler. Further, single mounting-interface 4 comprises one thermistor detector coupler 13 so the battery temperature can be monitored via a voltage governed by a thermistor voltage divider. Even further, the single mounting-interface 4 either comprises at least one universal asynchronous receiver-transmitter coupler pair in the form of a universal asynchronous receiver (RX)-coupler 14 and a universal asynchronous transmitter (TX)-coupler 15 or one synchronous serial interface coupler pair in the form of a serial clock (SCL) coupler 14 and a serial data (SDA) coupler 15 Lastly, there is a non-operational coupler 16. Thus, in this example, there are in total 10 couplers in the single mounting-interface. According to the above description, the effective number of operational couplers is 7. The single mounting-interface 4 further comprises a surface 17 to magnetically couple to the internal power supply unit 5 or the external power supply unit 7.

### Example 3—An Internal Power Supply Unit

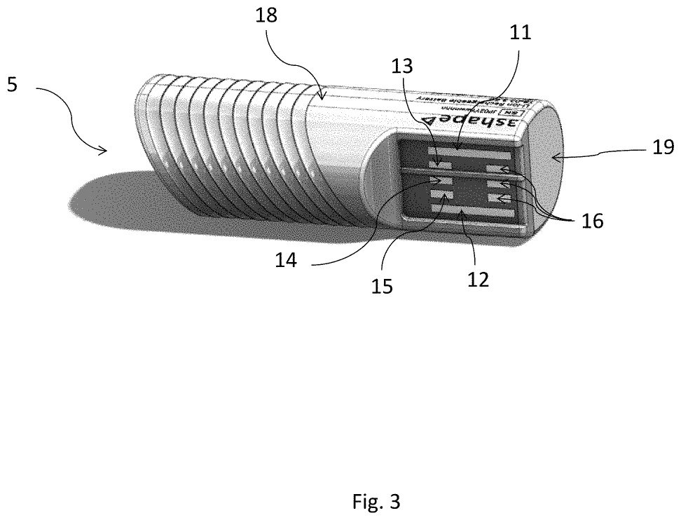

FIG. 3 shows an example of an internal power supply unit 5 according to the second aspect of the invention. FIG. 3. Shows an internal power supply unit 5 for powering the scanning device 1. The internal power supply is in this example an internal Li-Ion battery cell. The internal power supply unit 5 comprises a battery housing 18 dimensioned for being mounted into the single mounting-interface 4 of the scanning device 1.

The internal power supply unit 5 comprises one output voltage coupler 11, and a return connection coupler 12, the two couplers 11 and 12 together forming the power suppling path. Further, the internal power supply unit 5 comprises a first 12C signal (SCL) coupler 14 and a second 12C signal (SDA) coupler 15. Additionally, the internal power supply unit comprises a thermistor coupler 13, three non-operational couplers 16 and a surface 19 (in this example a metal plate) to magnetically couple to the single mounting-interface, specifically the surface 17 in the single mounting-interface (as shown in FIG. 2).

### Example 4—An External Power Supply Unit

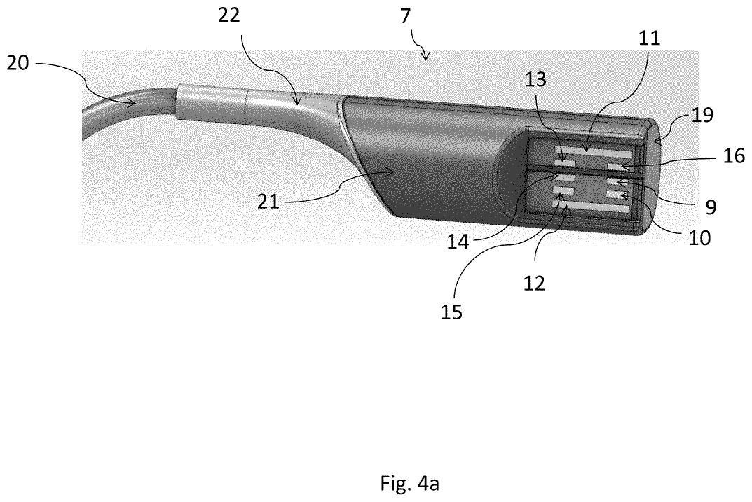

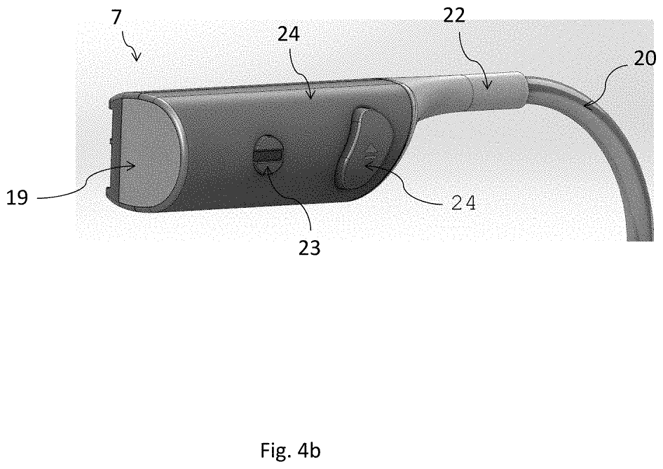

FIGS. 4a-4b show views of an external power supply unit according to the third aspect of the invention.

FIG. 4a shows a side-view of an external power supply unit 7 for powering the scanner device 1 through an ethernet cable 20, connected to an external power supply. The external power supply unit 7 comprises a plug 21 dimensioned for being mounted into the single mounting-interface 4 of the scanner device 1. The plug 21 comprises a soft cable relief 22. The external power supply unit 7 comprises one output voltage coupler 11, and a return connection coupler 12, the two couplers 11 and 12 together forming the power suppling path. Further, the internal power supply unit 7 comprises a first data coupler 9 for scanner communication through differential signaling (USB_N) and a second a data coupler 10 for scanner communication through differential signaling (USB_P). Additionally, the internal power supply 7 unit comprises a thermistor coupler 13 so the scanner device is able to identify the external power supply by pulling TS to GND (Signal voltage corresponds to a temperature of 220° C.). The internal power supply unit comprises a surface 19 (in this example a metal plate) to magnetically couple to the single mounting-interface, specifically the surface 17 in the single mounting-interface (as shown in FIG. 2). The external power supply unit comprises an additional device, wherein the additional device is a USB-to-Ethernet converter, such that an Ethernet cable 20 is mountable in the USB-to-Ethernet converter.

FIG. 4b shows a different side-view of the external power supply unit 7 for interlocking the unit 7 to the scanning device 1. The unit 7 comprises a snap lock mechanism 23 for interlocking the external power supply unit firmly to the frame of the scanning device within the single mounting-interface 4. Further, the external power supply unit 7 comprises a release button 24 for dismounting the external power supply from the scanning device 1.

### Example 5—A Device with the Internal or External Power Supply Unit

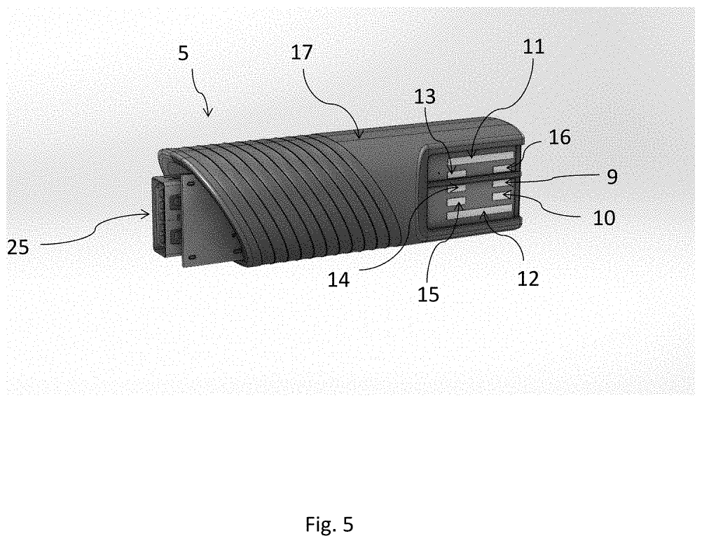

FIG. 5 shows an example of an internal power supply unit 5 according to the second aspect of the invention.

FIG. 5 Shows the internal power supply unit 5 for powering the scanner device 1 from an internal Li-Ion battery cell.

The internal power supply unit 5 comprises a battery housing 17 dimensioned for being mounted into the single mounting-interface 4 of the scanning device 1. The internal power supply unit 5 comprises one output voltage coupler 11, and a return connection coupler 12, the two couplers 11 and 12 together forming the power suppling path. Further, the internal power supply unit 5 comprises a first I2C signal (SCL) coupler 14 and a second I2C signal (SDA) coupler 15. Additionally, the internal power supply unit comprises a thermistor coupler 13, one non-operational coupler 16. Additionally, the internal power supply unit 5 comprises a first data coupler 9 for scanner communication through differential signaling (USB_N) and a second a data coupler 10 for scanner communication through differential signaling (USB_P). The internal power supply unit 5 comprises a USB feedthrough interface 25 in the rear of the unit 5 configured to bypass the battery cell when the scanning device 1 detects a wired connection, such that the scanner device may be powered directly via a USB-cable. In this way, the internal power supply unit 5 comprises the feedthrough interface 25 to mount the external power supply unit 7.

### Example 6—A Second Embodiment of the Device

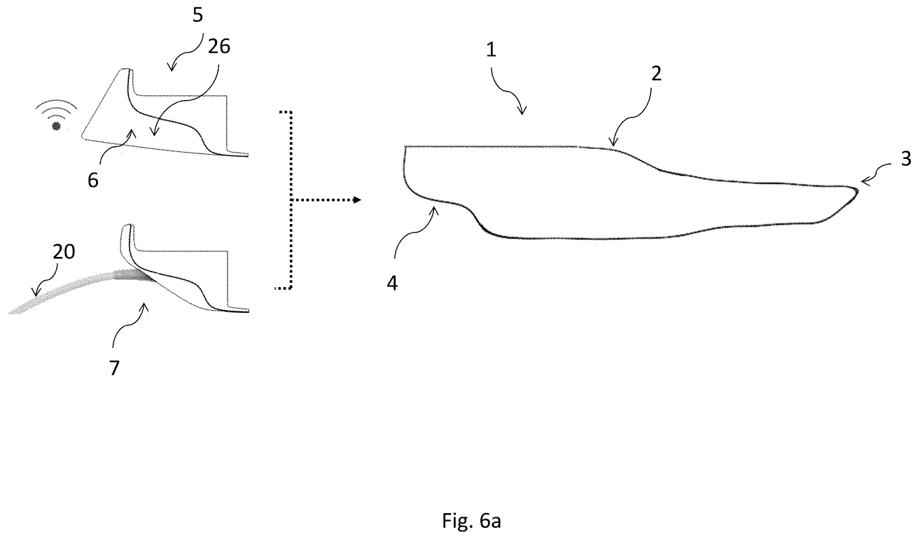

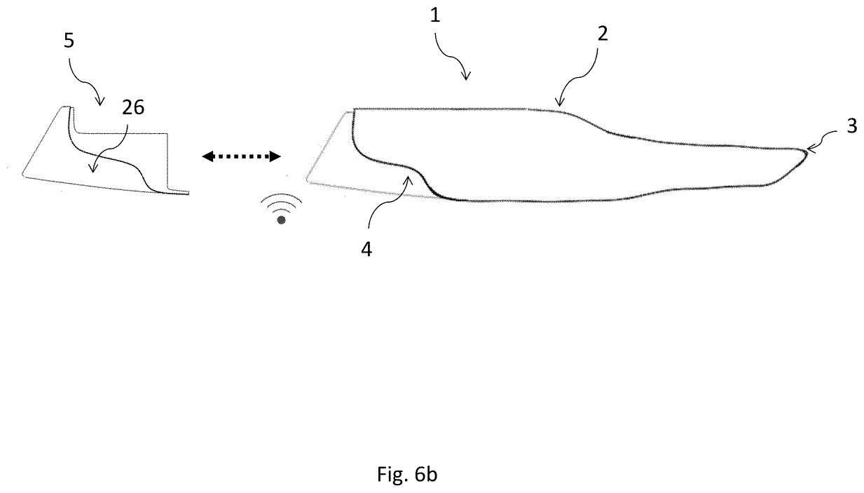

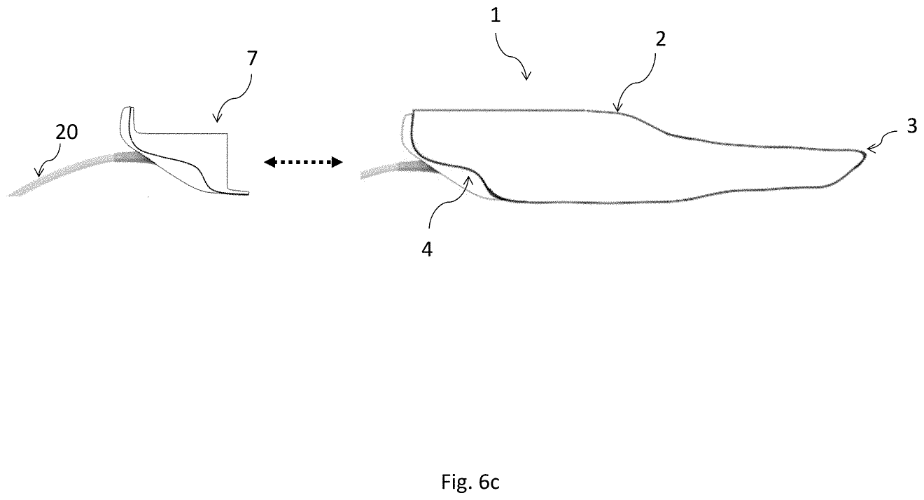

FIGS. 6a-6c shows an example of an alternative configuration of the scanning device 1 and single mounting-interface 4. The scanning device 1 is modular constructed and enables a configuration of internal powered and wireless data transfer or externally powered and wired data transfer.

FIG. 6a shows a device 1 for scanning a human intra-cavity, comprising: a housing 2 comprising a part 3 connected to the housing 2, the part 3 dimensioned for being inserted into said human intra-cavity, a single mounting-interface 4 on the housing 2, the single mounting-interface 4 configured to removably mount: an internal power supply unit 5 for powering the device from an internal power supply 6 within the internal power supply unit, or an external power supply unit 7 for powering the device from an external power supply outside the external power supply unit. As can be seen, the device is configured for being changed between two power-operation modes: an internal power-operation mode, where the device 1 is directly powered by the internal power supply unit 5 via the single mounting-interface 4, and an external power-operation mode, where the device 1 is directly powered by the external power supply unit 7 via the single mounting-interface 4. The internal power supply unit 5 is in the form of a wireless add-on module for powering the device 1 from an internal power supply 6 within the wireless add-on module and facilitates wireless data transfer through an internal wireless module 26. The external power-supply unit 7 is in the form of a wired add-on module for powering the device from an external power supply 8 outside the wired add-on module and facilitates wired data transfer through a wired interface 20, i.e. a cable.

FIG. 6b shows the scanning device 1 with the wireless add-on module 5 mounted in the single mounting-interface 4 of the device 1. The single mounting-interface 4 is configured as an RGMII or PCI-express interface.

The wireless add-on module 5 is dimensioned to fit into the single mounting-interface 4 and configured with a Li-ion battery cell, embedded Linux and USB Wi-Fi adapter such that the wireless add-on is considered a deployable Wi-Fi bridge. The wireless add-on module 5 comprises the internal wireless module 26 and enables the scanning device 1 to be operated in a fully wireless mode of both power supply and data transfer.

FIG. 6c shows the scanning device 1 with the wired add-on module 7 mounted in the single mounting-interface 4 of the device 1. The single mounting-interface 4 is configured as a RGMII or PCI-express or USB interface. The wired add-on module 7 is dimensioned to fit into the single mounting-interface 4 and configured with an Ethernet feedthrough and/or PHY for providing wired power and data transfer.

### Example 7—Prior Art

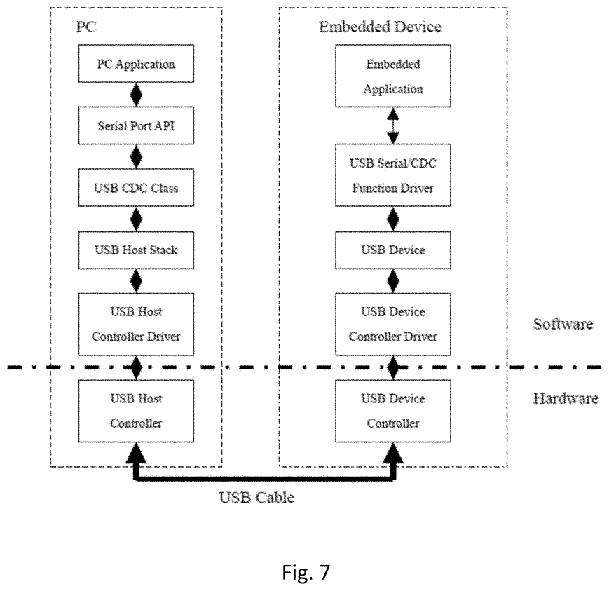

FIG. 7 shows an example of prior art of wired communication with a USB-cable between an embedded device and a PC. FIG. 7 is shown for comparative reasons with the present invention.

### Example 8—Prior Art

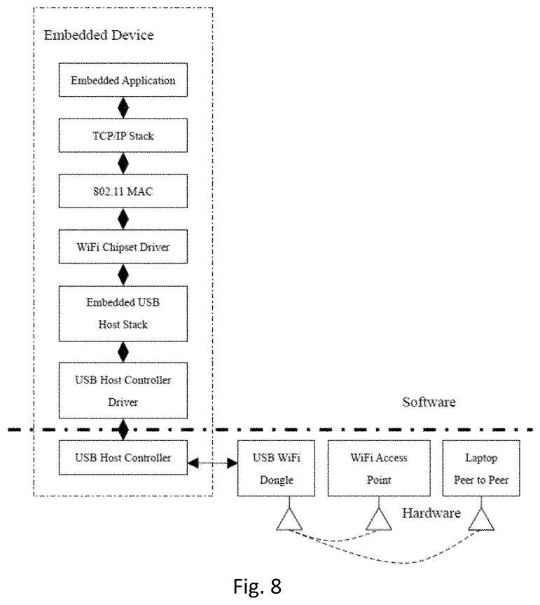

FIG. 8 shows an example of wireless communication with USB to WiFi between an embedded device and a PC. FIG. 8 is shown for comparative reasons with the present invention.

### Example 9—an Example of a Host Controller with Wired Connection

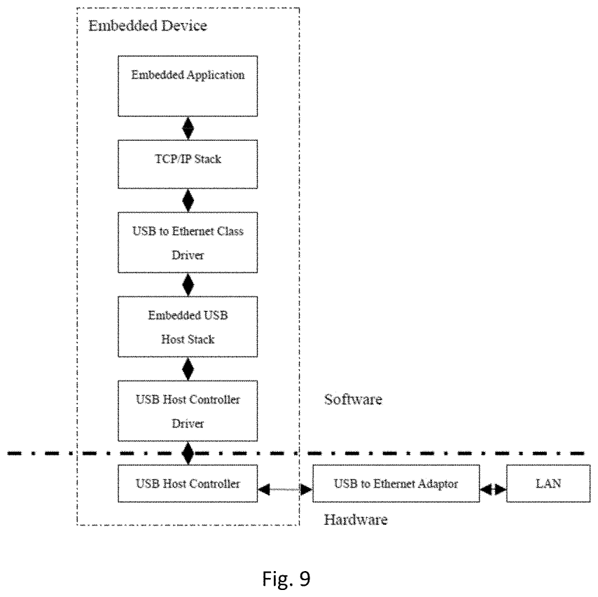

FIG. 9 shows an example of how the scanning device according to the present invention communicates over a wire between the scanning device (the embedded device and a PC) using a host controller located on the scanning device. The USB to Ethernet class driver interfaces the USB host stack to a TCP/IP stack in the scanning device.

### LIST OF FIGURE REFERENCE NUMBERS

1. Scanning device
2. Scanning housing
3. Scanning part
4. Single mounting-interface
5. Internal power supply unit
6. Power storing/generating means within the internal power supply
7. External power supply
8. Power supply for powering the external power supply unit
9. USB_N data coupler
10. USB_P data coupler
11. Input voltage coupler
12. Return path voltage coupler
13. Thermistor detector coupler
14. Universal asynchronous receiver-transmitter (RX) or I2C coupler
15. Universal asynchronous receiver-transmitter (TX) or I2C coupler
16. Non-operational coupler
17. Surface to magnetically couple to the internal power supply unit or the external power supply unit.
18. Battery housing
19. Surface to magnetically couple to the single mounting-interface.
20. Cable, ethernet cable
21. Plug
22. Soft cable relief
23. Lock mechanism
24. Release button
25. Feedthrough interface
26. Wireless module

Further details are provided in the below items.

### Items:

1. A device for scanning a human intra-cavity, comprising:
   a housing comprising a part connected to the housing, the part dimensioned for being inserted into said human intra-cavity,
   a single mounting-interface on the housing, the single mounting-interface configured to removably mount:
      i. an internal power supply unit for powering the device from an internal power supply within the internal power supply unit, or
      ii. an external power supply unit for powering the device from an external power supply outside the external power supply unit,
      the device configured for being changed between two power-operation modes:
      i. an internal power-operation mode, where the device is directly powered by the internal power supply unit via the single mounting-interface, and
      ii. an external power-operation mode, where the device is directly powered by the external power supply unit via the single mounting-interface.
2. The device according to item 1, wherein the device is further configured for being changed between two data-operation modes:
   i. a wireless data-operation mode, where the device transfers data in a form of wireless data signals to a wireless module, and
   ii. a wired data-operation mode, where the device transfers data in a form of wired data signals to the single mounting-interface.
3. The device according to items 1-2, wherein the device is further configured for providing any combination of the two power-operation modes and the two data-operation modes.
4. The device according to items 1-2, wherein the device is further configured for being changed between two fully-operation modes:
   i. a fully wireless-operation mode, where the device is in the internal power-operation mode and the wireless data-operation mode, and
   ii. a fully wired-operation mode, where the device is in the external power-operation mode and the wired data-operation mode.
5. The device according to any of the preceding items, wherein the single mounting-interface comprises:
   at least one data coupler,
   at least one voltage coupler, and
   at least one detector coupler.
6. The device according to item 5, wherein the single mounting-interface further comprises at least one universal asynchronous receiver-transmitter coupler pair in the form of a universal asynchronous receiver coupler and a universal asynchronous transmitter coupler, or one synchronous serial interface coupler pair in the form of a serial clock coupler and a serial data coupler.
7. The device according to any of the preceding items, wherein the single mounting-interface comprises a part that geometrically matches a part of the internal power supply unit and geometrically matches a part of the external power supply unit.
8. The device according to any of the preceding items, wherein the single mounting-interface comprises a surface to magnetically couple to the internal power supply unit and/or the external power supply unit.
9. The device according to any of the preceding items, wherein the single mounting-interface comprises a mechanical locking mechanism to couple to the internal power supply unit and/or the external power supply unit.
10. The device according to any of the preceding items, wherein the device further comprises an acquisition unit configured for acquiring raw data of the human intra-cavity.
11. The device according to item 10, wherein the device further comprises a first processing unit linked to the acquisition unit and configured to process the raw data into processed data.
12. The device according to items 2 and 11, wherein the device further comprises a second processing unit coupled to the acquisition unit and linked to the wireless module and the single mounting-interface such that the processed data is send to the wireless module in the wireless data-operation mode and the processed data is send to the single mounting-interface in the wired data-operation mode.
13. The device according to item any of the items 11-12, wherein the first processing unit and/or the second processing unit is configured to run software allowing the device to act as a host for the wireless module and to act as a host for an additional device in the external power supply unit.
14. The device according to item 2, wherein device is further configured to disable the wireless module upon the external power supply unit being mounted in the single mounting-interface and/or upon the internal power supply unit being dismounted the from the single mounting-interface.
15. The device according to item 2, wherein device is further configured to enable the wireless module upon the internal power supply unit being mounted in the single mounting-interface or upon the external power supply unit being mounted in the single mounting-interface.
16. An internal power supply unit for a device according to any of the items 1-15, wherein the internal power supply unit is a battery.
17. The internal power supply unit according to item 16, wherein the internal power supply unit comprises:
   at least one voltage coupler, and
   at least one detector coupler.
18. The internal power supply unit according to item 17, wherein the internal power supply unit further comprises at least one at least one universal asynchronous receiver-transmitter coupler pair in the form of a universal asynchronous receiver coupler and a universal asynchronous transmitter coupler, or one synchronous serial interface coupler pair in the form of a serial clock coupler and a serial data coupler.
19. The internal power supply unit according to any of the items 16-18, wherein the internal power supply comprises a surface to magnetically couple to the single mounting-interface.
20. The internal power supply unit according to any of the items 16-19, wherein the internal power supply unit comprises an interface to mount the external power supply unit.
21. The internal power supply unit according to any of the items 16-20, wherein the internal power supply unit comprises a wireless module.
22. An external power supply unit for a device according to any of the items 1-15, wherein the external power supply unit is configured to be wired to an external power supply.
23. The external power supply unit according to item 22, wherein the external power supply unit comprises:
   at least one data coupler,
   at least one voltage coupler, and
   at least one detector coupler.
24. The external power supply unit according to item 23, wherein the external power supply unit further comprises at least one universal asynchronous receiver-transmitter coupler pair.
25. The external power supply unit according to any of the items 22-24, wherein the external power supply unit comprises an additional device, wherein the additional device is a USB-to-Ethernet converter, such that an Ethernet cable is mountable in the USB-to-Ethernet converter.
26. The external power supply according to any of the items 22-25, wherein the external power supply unit comprises a surface to magnetically couple to the single mounting-interface.
27. The external power supply unit according to any of the items 22-26, wherein the external power supply unit comprises a locking mechanism to couple to the single mounting-interface.
28. The external power supply unit according to any of the items 22-27, wherein the external power supply comprises an interface to mount the internal power supply unit.
29. An intra-cavity scanning system, comprising:
   the device according to any of the items item 1-15; and
   the internal power supply unit according to any of the items 16-21; and/or
   the external power supply unit according to any of the items 22-28.

1. A device for scanning a human intra-cavity, comprising:
a housing comprising a part connected to the housing, the part dimensioned for being inserted into said human intra-cavity,
a single mounting-interface on the housing, the single mounting-interface configured to removably mount:
i. an internal power supply unit for powering the device from an internal power supply within the internal power supply unit, or
ii. an external power supply unit for powering the device from an external power supply outside the external power supply unit,
the device configured for being changed between two power-operation modes:
i. an internal power-operation mode, where the device is directly powered by the internal power supply unit via the single mounting-interface, and
ii. an external power-operation mode, where the device is directly powered by the external power supply unit via the single mounting-interface,
wherein the device is further configured for being changed between two data-operation modes:
i. a wireless data-operation mode, where the device transfers data in a form of wireless data signals to a wireless module, and
ii. a wired data-operation mode, where the device transfers data in a form of wired data signals to the single mounting-interface, wherein
the device comprises a host controller, the host controller configured to transfer the data in both the wireless data-operation mode and the wired data-operation mode.

2. The device according to claim 1, wherein the device is further configured for being changed between two fully-operation modes:
iii. a fully wireless-operation mode, where the device is in the internal power-operation mode and the wireless data-operation mode, and
iv. a fully wired-operation mode, where the device is in the external power-operation mode and the wired data-operation mode.

3. The device according to claim 1, wherein the single mounting-interface comprises:
at least one data coupler,
at least one voltage coupler, and
at least one detector coupler.

4. The device according to claim 3, wherein the single mounting-interface further comprises at least one universal asynchronous receiver-transmitter coupler pair in the form of a universal asynchronous receiver coupler and a universal asynchronous transmitter coupler, or one synchronous serial interface coupler pair in the form of a serial clock coupler and a serial data coupler.

5. The device according to claim 1, wherein the single mounting-interface comprises a part that geometrically matches a part of the internal power supply unit and geometrically matches a part of the external power supply unit.

6. The device according to claim 1, wherein the device further comprises an acquisition unit configured for acquiring raw data of the human intra-cavity.

7. The device according to claim 6, wherein the device further comprises a first processing unit linked to the acquisition unit and configured to process the raw data into processed data in the form of 3D data.

8. The device according to claim 1, wherein the device further comprises a second processing unit coupled to the acquisition unit and linked to the wireless module and the single mounting-interface such that the processed data is send to the wireless module in the wireless data-operation mode and the processed data is send to the single mounting-interface in the wired data-operation mode.

9. The device according to claim 1, wherein the host controller, together with a host controller driver, acts as a host for the wireless module and acts as a host for an additional device in the external power supply unit.

10. The device according to claim 1, wherein the host controller is a USB host controller.

11. An external power supply unit for a device according to claim 1, wherein the external power supply unit is configured to be wired to an external power supply.

12. The external power supply unit according to claim 11, wherein the external power supply unit comprises:
at least one data coupler,
at least one voltage coupler, and
at least one detector coupler.

13. The external power supply unit according to claim 12, wherein the external power supply unit further comprises at least one universal asynchronous receiver-transmitter coupler pair.

14. The external power supply unit according to claim 11, wherein the external power supply unit comprises an additional device, wherein the additional device is a USB-to-Ethernet converter, such that an Ethernet cable is mountable in the USB-to-Ethernet converter.

15. An intra-cavity scanning system, comprising:
the device according to claim 1; and
the internal power supply unit; or
the external power supply unit, wherein the external power supply unit is configured to be wired to an external power supply.

16. An intra-cavity scanning system, comprising:
the device according to claim 1; and
the internal power supply unit; and
the external power supply unit, wherein the external power supply unit is configured to be wired to an external power supply.
